(19) United States
(12) Patent Application Publication
(10) Pub. No.: US 2020/0179599 A1
(43) Pub. Date: Jun. 11, 2020

(54) APPARATUS AND METHOD FOR CREATING A SOLID STATE CLOSED LOOP ARTIFICIAL PANCREAS DEVICE USING LED ARRAYS FOR GLUCOSE VARIABILITY CONTROL

(71) Applicants: Rama Madugula, Royal Oak, MI (US); Anup Sabharwal, Miami, FL (US); Sankar Majeti, Rochester Hills, MI (US)

(72) Inventors: Rama Madugula, Royal Oak, MI (US); Anup Sabharwal, Miami, FL (US); Sankar Majeti, Rochester Hills, MI (US); Murty Madugula, Rochester Hills, MI (US)

(73) Assignee: Midwest Business Associates, Inc., Rochester Hills, MI (US)

(21) Appl. No.: 16/210,099

(22) Filed: Dec. 5, 2018

(51) Int. Cl. A61M 5/172; A61B 5/145; A61M 5/142; A61B 5/1455

(52) U.S. Cl. CPC A61M 5/1723; A61B 5/14532; A61B 5/6898; A61B 5/1455; A61M 5/14244

(57) ABSTRACT

Disclosed is a light-based artificial pancreas system for dispensing drug formulations to a diabetic patient that monitors the status of the patient to ensure that blood glucose levels remain within a customizable range. Drug formulations dispensable by the system contain at least one photoswitchable compound, including insulin, whose dosage amounts are controllable by light energy and based on both light wavelength and light intensity. The system dispenses drug formulations based on light generated by a set of LED arrays that includes blue or violet LED's; the light is detected by photoreceivers within a drug reservoir that contains the drug formulations. The system is designed such that multiple drug formulations used simultaneously are each controlled by a separate LED array and by separate photoreceivers operating at separate wavelengths such that the drug formulations, LED arrays, photoreceivers and wavelengths do not interfere with each other.

### CROSS REFERENCE TO RELATED APPLICATIONS

The present invention is a continuation-in-part (CIP) of U.S. patent application Ser. No. 15/247,796, filed 25 Aug. 2016, entitled “Light-Based Drug Dispensing System to Simulate the Operation of a Human Pancreas”, by Rama Madugula, et al., which is, in turn, a continuation-in-part (CIP) of U.S. patent application Ser. No. 14/724,854 (abandoned), filed 29 May 2015, entitled “System for Medical Device Software Alert Hierarchy Management”, by Rama Madugula, et al.

### BACKGROUND OF THE INVENTION

### a. Field of the Invention

The present invention generally pertains to an artificial pancreas and more particularly to a solid state closed loop device that functions as an artificial pancreas.

### b. Description of the Background

Patients with diabetes often require insulin to control their blood sugar. In many cases, insulin is pumped into a patient's body through an electromechanical pump that may eventually clog, need calibration, break down or simply wear out.

In recent years, a class of azobenzenes has been developed that change state, or are “photoswitchable”, when exposed to a specific wavelength of light. These azobenzenes may be combined with, or located within, other chemical compounds, such as insulin, sulfonylureas, glucagon or other diabetic pharmaceutical compounds, to activate those compounds and make them also photoswitchable. Thus, controls on the intensity, wavelength, duration or duty-cycle of a light source may be used to release controlled dosages of photoswitchable drug formulations. These controls allow such drug formulations to be released into the human body without using an electromechanical pump that is prone to failure.

It would therefore be advantageous to have a solid state device as part of a system that monitors the health of a diabetes patient and uses and controls light to dispense insulin or other drug compounds to a patient without using a pump, thus avoiding the issues associated with using a pump. Such a device could be a solid-state artificial pancreas that uses closed-loop feedback control (such as proportional-integral-derivative [PID] control, model predictive control [MPC] or Linear Quadratic Ganussian control [LQG] within a wearable computer with sensor inputs, a battery pack, an LED array for dispensing photoswitchable compounds and a display/speaker for output, to keep a patient's blood glucose level within a range specified by a physician and provide alerts when the glucose level is outside of that range. Such control requires a continuous glucose monitor to measure blood glucose level and a wearable computer with a program that uses closed-loop feedback to keep blood glucose levels within a range specified by a physician.

### SUMMARY OF THE INVENTION

The present invention overcomes the disadvantages and limitations of the prior art by providing a solid-state artificial pancreas system that uses a closed-loop feedback control algorithm (such as PID, MPC or LQG) within a wearable computer to keep a patient's blood glucose level within a range specified by a physician.

The advantages of the present invention are the avoidance of problems inherent in electromechanical pumps, which are prone to wear and tear, clogging, dose calibration issues and pump breakdown.

### BRIEF DESCRIPTION OF THE DRAWINGS

In the drawings,

FIG. 1 is a schematic representation of the various elements of the present invention.

FIG. 2 is an illustration of an operational flowchart for one embodiment of the present invention.

### DETAILED DESCRIPTION OF THE INVENTION

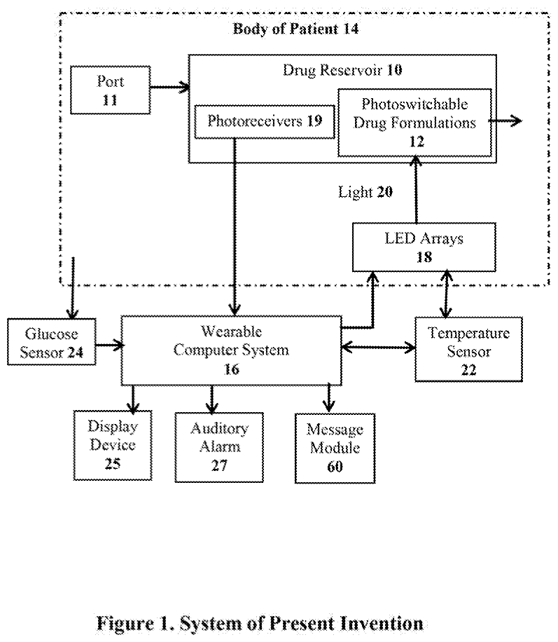

FIG. 1 is a drawing of the system of the present invention, which includes a drug reservoir 10 that may receive and hold at least one photoswitchable drug formulation 12, in addition to compounds that may be given orally, that may be given to the body of a patient 14 (indicated by a dashed box). The drug reservoir 10, re-supplied as necessary by adding a compound through a port 11 in the body 14, may be subcutaneous or intramuscular or located within the abdominal cavity of the body of a patient 14, adjacent to the patient's organs. In some embodiments, the system may alert the patient to take drug formulations orally 12 (not shown in FIG. 1).

In typical embodiments, the photoswitchable drug formulations 12 may each contain an azobenzene or other synthetic molecular switch compound whose photoswitchability can be controlled by a wearable computer system 16. The drug reservoir 10 may hold at least one photoswitchable drug formulation 12 that may be released into the body of the patient 14 in controlled doses via controlled amounts of light energy from at least one set of LED arrays 18. In optimum embodiments, the LED arrays 18 may be adjacent to the drug reservoir 10, and both may be located either under the skin, within the abdominal cavity (i.e., next to the pancreas or liver), or within a muscle of the body of the patient 14.

The wearable computer system 16 may include sensor inputs, a battery pack, an LED array for dispensing photoswitchable compounds and a display/speaker for output. The wearable computer system 16 may be set by a physician to provide hierarchical alerts and feedback and to activate and control the sets of LED arrays 18 at specific, calibratable intervals (such as, every minute or every 5 minutes) to generate light 20 at specified wavelengths, duty cycle or duration that may be received by a set of photoreceivers 19 within the drug reservoir 10 when an LED within the LED array 18 is activated. The photoreceivers 19 ensure that the light 20 reaches the photoswitchable drug formulations 12 within the drug reservoir 10 so that the photoswitchable drug formulations 12 may be dispensed. If the wearable computer system 16 activates the LED array 18 but the photoreceivers 19 do not detect the light, the wearable computer system 16 may set a warning message or alert, display an appropriate diagnostic failure message, and possibly enter a “safe mode.” In optimum embodiments, each set of LED's within an LED array 18 is set to its own individual wavelength and can thus only activate one of several available photoswitchable drug formulations 12 when activated by the wearable computer system 16.

The wearable computer system 16 may also be set by a physician to control the parameters of the set of LED arrays 18, including the pulse width modulation duty cycle and the maximum amount of LED activation within a given time period. The wearable computer system 16 may further be set by a physician to monitor other parameters as needed, including the temperature range of the LED arrays 18 as registered by the temperature sensor 22, the patient's heart rate and/or ketone levels, the amount of medication in the drug reservoir 10, and/or any other parameters deemed necessary by the physician, as well as the range of glucose levels registered by the glucose sensor 24 that should deactivate all LED's in the LED arrays 18. As a safety mechanism, the wearable computer system 16 may also be set by a physician to limit the total amount of time the LED arrays 18 may be activated within a defined period to protect a patient from an overdose of a photoswitchable drug formulation 12.

In some embodiments, the LED arrays 18 may control the release of the photoswitchable drug formulations 12 by being active for enough time to allow a desired dose to be dispensed. For example, if the drug reservoir 10 is designed to release one unit of insulin for every second that an LED is activated, and a patient requires five units of insulin, the LED corresponding to insulin may be activated for five seconds by the wearable computer system 16 and then turned off.

In other embodiments, a desired dose may be dispersed by controlling the intensity of light output from the LED arrays 18 by pulse width modulation. For example, if the drug reservoir 10 is set to always release 10 units of insulin when exposed to 10 seconds of light at 100% intensity, the wearable computer system 16 may activate the LED corresponding to insulin at 50% intensity for 10 seconds, thus keeping the signal duration unchanged but still delivering only five units of insulin.

Information from the wearable computer system 16 may be viewed on a display device 25 and output to a message module 60 to allow notification of the patient, family members and caregivers of any parameter outside established ranges. Any parameter outside established ranges may cause the wearable computer system 16 to generate an auditory alarm 27 that something in the system of FIG. 1 needs to be repaired. An auditory alarm 27 may also be generated to indicate a problem when the photoreceivers 19 detect that light from the LED arrays 18 is not reaching the photoswitchable drug formulations 12 within the drug reservoir 10.

In optimum embodiments of the present invention, the LED's in the LED arrays 18 may be colored (especially blue or violet), and each photoswitchable drug formulation 12 may be controlled by a separate LED array 18 and photoreceivers 19 that operate at different wavelengths such that the photoswitchable drug formulations 12, LED arrays 18, photoreceivers 19 and light 20 do not interfere with each other.

The photoswitchable drug formulations 12 may include (but are not limited to): a diabetes drug formulation, insulin, glucagon, ketones, D10, D50, an anti-inflammatory compound, a chemotherapeutic compound, growth hormones, contraceptives, an analgesic compound, or any other drug formulation deemed medically beneficial for a patient. Diabetes drug formulations usable with the present invention may include Metformin, a sulfonylurea compound (such as JB253, gliburide, glipizide or glimepiride), meglitinides, thiazolidinediones, DPP-4 inhibitors, GLP-1 receptor agonists or SGLT2 inhibitors.

Various sensors that are used to help determine and measure blood glucose levels and other body parameters include, but are not limited to, a body temperature sensor 22; a continuous glucose monitoring sensor 24; a ketone meter; an Hba1C sensor, a heart rate monitor; a blood pressure monitor; an activity monitor (such as a step counter); and a swallow sensor (to detect food being swallowed), and may be connected to the patient 14 either inside or outside of the patient's body. All sensor outputs may be provided to the wearable computer system 16, along with the outputs of the photoreceivers 19. Both the drug reservoir 10 and sets of LED arrays 18 may be located either inside or outside of the patient's body 14; FIG. 1 shows an embodiment where both are inside the body of a patient 14.

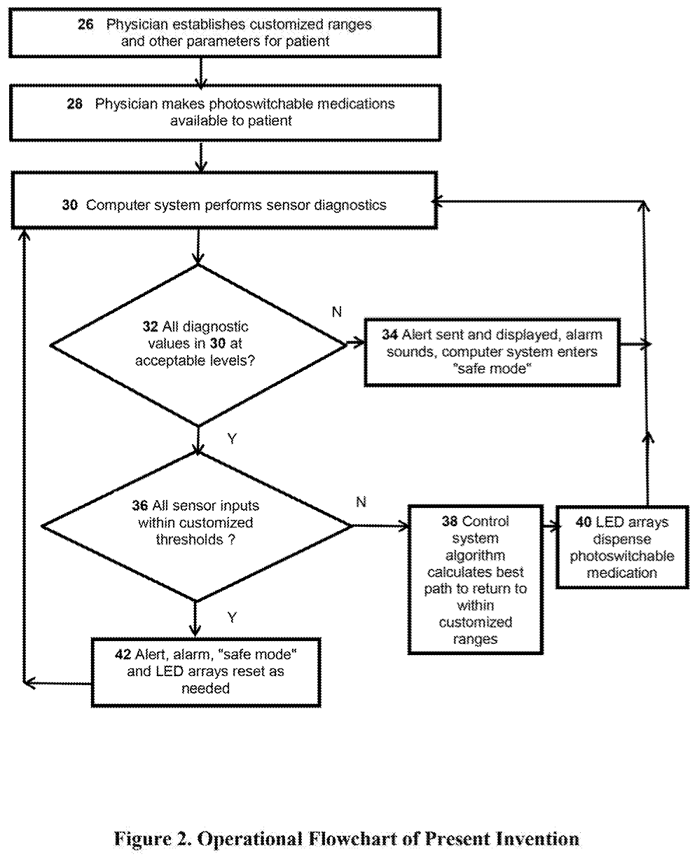

FIG. 2 illustrates an operational flowchart for one embodiment of the present invention. A physician establishes a range of allowable temperatures for the LED arrays 18 such that the LED arrays 18 do not burn the patient 14. The physician also establishes a range of “normal” glucose levels 26 individualized for the patient 14, along with a range for any other sensors that may be used in the wearable computer system 16 (such as a heart monitor, body temperature sensor, ketone sensor, blood pressure sensor, or a sensor monitoring the amount of medication in the drug reservoir 10).

The physician 28 then makes the photoswitchable drug formulations 12 (referring to FIG. 1) available to the patient by placing the photoswitchable drug formulations 12 in the drug reservoir 10 through the drug reservoir port 11. As long as the photoreceivers 19 are working properly, the drug reservoir 10 contains sufficient photoswitchable drug formulations 12, and the patient's temperature range, glucose level and other parameters are within normal ranges as detected by the temperature sensor 22, glucose sensor 24 and other sensors in the system, the present invention may remain in a quiescent state 30, continually monitored by the wearable computer system 16. The physician always has the option to use regular diabetic pharmaceutical compounds administered orally, based on alerts and messaging from the control program to the patient to prompt them to take the medicines orally.

As typical in closed-loop feedback systems, the wearable computer system 16 may loop many times each second. During each loop, the wearable computer system 16 may send out a hierarchical set of alerts and messages while performing these functions:

1) Reads and performs diagnostic checks on system components whose values must stay at a minimum level 32, including some sensors, the LED array 18, the system battery level and the level of the drug reservoir 10. Also reads and performs diagnostic checks on system components whose values must stay within operational ranges, including temperature sensor 22, glucose sensor 24, and heart rate sensor. If the computer program detects a component value outside the acceptable operational range, the computer program 34 alerts a user and enters “safe mode.” For example, whenever the photoreceivers 19 detect a lack of light to the photoswitchable drug formulations 12, the drug reservoir 10 needs more drug formulation, the LED array 18 is hot enough to burn the patient's skin, or a sensor level is outside of the range calibrated by the physician, the computer program 32 may detect the situation and 34 send an alert to the display device 25 (referring to FIG. 1) and message module 60, send an alert to the auditory alarm 27 (referring to FIG. 1) to create a sound, and put the computer program into a “safe mode” until minimum levels are again reached.

2) Reads sensor inputs 36. If a blood glucose level is detected outside the customized range, either higher or lower than threshold values, the wearable computer system 16 may send real-time alerts to the user and/or caregiver, via display or wireless transmission, that a hypoglycemic or hyperglycemic event has taken place. The wearable computer system 16 then proceeds to

3) Calculate values, using control algorithms, and store these values as necessary. The wearable computer system 16 makes calculations based on sensor inputs and uses a well-known control system algorithm (such as, but not limited to, PID, MPC or LQG control) 38 to determine the best and fastest way to bring the value back within the customized range. For example, if the glucose sensor 24 reading is higher than the maximum acceptable value, the computer program may calculate the amount of insulin or other drug formulations, such as sulfonylureas, to be released.

4) (as necessary) Performs actions (i.e., sending activation commands to one or more specific LED arrays 38) and sends messages and alerts. For example, if glucose level is below the customized level, the wearable computer system 16 may activate a combination of LED arrays 38 to generate light at specified wavelengths and intensities corresponding to at least one photoswitchable drug formulation 12, typically a glucose-boosting compound such as D10, D50 or glucagon.

The specific wavelengths and intensities of the light 20 (referring to FIG. 1) activate the photoswitchable drug formulations 12, which are then delivered to the patient in an appropriate dosage calculated by the physician and wearable computer system 16 from data provided by the glucose sensor 24 and other sensors in the system. The photoswitchable drug formulations 12 may be administered until the glucose sensor and other sensors indicate 36 that the patient's glucose and other levels are again within the “normal” range. The wearable computer system 16 then deactivates the LED arrays 40, which turns off the LED's and ends the dosage of the photoswitchable drug formulations 12.

The photoreceivers 19 may again detect light being sent to the photoswitchable drug formulations 12 and the drug reservoir 10 may be replenished through the drug reservoir port 11 as needed, at which point the wearable computer system 16 turns off all alerts 42 to the display device 25, message module 60 and auditory alarm 27.

The wearable computer system 16 may also calculate values over a time period and send these analytics to a physician or caregiver at regular intervals. These non-real time, non-emergency alerts may help the physician to monitor a large patient population on a weekly or monthly basis by concentrating on calculated values like average blood glucose level, average activity, average HbA1C level, and average amount of insulin dispensed over a day, week, 1-month or 3-month period.

The wearable computer system 16 may further gather data and statistics on the operation of the LED arrays 18, or on the number of interventions needed to maintain glucose variability control and transmit this information back to the physician or to a manufacturer.

5) Returns to the Top of the Loop (Step 1 Above)

Note that even in cases where the patient 14 has received at least one drug formulation 12 orally, the light 20 may still control the photoswitchable drug formulation within the body of the patient 14 by having the wearable computer system 16 direct operation of the LED arrays 18 as described above.

When insulin or a sulfonylurea photoswitchable drug formulation, along with a continuous glucose monitoring sensor, is used in the present invention, the present invention provides a solid state LED array closed-loop “artificial pancreas” that uses feedback and allows controlled release of insulin to a patient. This solid state artificial pancreas is superior to an electromechanical pump that may clog, need calibration, break down or simply wear out.

In all embodiments of the present invention, alerts may be sent by common electronic communications methods, including wireless methods, that may include wi-fi, e-mail, texting, cell phone and Bluetooth. Such alerts may be seen on a remote device such as a smartphone, wrist watch or other display in a remote location and may notify all interested parties, such as a patient, caregiver, physician and hospital, of a patient's health issues.

The foregoing description of the invention has been presented for purposes of illustration and description. It is not intended to be exhaustive or to limit the invention to the precise form disclosed, and other modifications and variations may be possible in light of the above teachings. The embodiment was chosen and described in order to best explain the principles of the invention and its practical application to thereby enable others skilled in the art to best utilize the invention in various embodiments and various modifications as are suited to the particular use contemplated. It is intended that the appended claims be construed to include other alternative embodiments of the invention except insofar as limited by the prior art.

What is claimed is:

1. A solid state closed loop artificial pancreas device that uses LED arrays to monitor glucose levels for a patient, comprising:
at least one sensor, connected inside the body or outside the body of said patient, that measures glucose levels of said patient;
at least one sensor to monitor the physical condition of said artificial pancreas device;
at least one medical device connected to said patient that affects at least one health parameter of said patient and can be affected by closed-loop feedback;
at least one photoswitchable drug formulation whose structure and dosage amounts are controllable by light energy and based on both light wavelength and light intensity;
a drug reservoir that receives and holds said photoswitchable drug formulation, can be resupplied through a port, is connected inside the body or outside the body of said patient, and includes at least one set of photoreceivers that detects light from at least one set of LED arrays;
a wearable computer system that uses closed-loop feedback from data provided by said sensor and said medical device to calculate values over a time period and send said values to a physician at regular intervals, to provide hierarchical alerts whenever said sensor or said medical device detects a value outside the established range for said patient or whenever the glucose level of said patient is outside customized values for said patient, and uses a control system algorithm to determine the best and fastest way to bring a value back within range;
at least one set of said LED arrays, controllable by said wearable computer system's setting the pulse width modulation duty cycle or setting the maximum amount of LED activation within a given time period and connected inside the body or outside the body of said patient, whose LED's control the dosage of said photoswitchable drug formulation given to said patient whenever the glucose level of said patient is outside customized values for said patient;
a set of photoreceivers within said drug reservoir that passes light provided by said LED arrays to said photoswitchable drug formulation so that said photoswitchable drug formulation is dispensed to said patient whenever the glucose level of said patient is outside customized values for said patient; and,
at least one remote electronic display that can use wireless communications methods to receive and display said hierarchical alerts.

2. The solid state closed loop artificial pancreas device of claim 1, wherein said photoswitchable drug formulation affects glucose levels of said patient.

3. The solid state closed loop artificial pancreas device of claim 2, wherein said photoswitchable drug formulation is a diabetes drug formulation, insulin, glucagon, ketones, D10, D50, an anti-inflammatory compound, a chemotherapeutic compound, growth hormones, contraceptives or an analgesic compound.

4. The solid state closed loop artificial pancreas device of claim 3, wherein said diabetes drug formulation is Metformin, a sulfonylurea compound, a meglitinide, a thiazolidinedione, a DPP-4 inhibitor, a GLP-1 receptor agonist or an SGLT2 inhibitor.

5. The solid state closed loop artificial pancreas device of claim 4, wherein said sulfonylurea compound is JB253, gliburide, glipizide or glimepiride.

6. The solid state closed loop artificial pancreas device of claim 1, wherein the LED's within said LED arrays are colored blue or violet.

7. The solid state closed loop artificial pancreas device of claim 1, wherein said computer system controls said LED arrays and said photoreceivers to operate at different wavelengths of light such that said photoswitchable drug formulations, said LED arrays, said photoreceivers and said wavelengths of light do not interfere with each other.

8. The solid state closed loop artificial pancreas device of claim 1, wherein said control system algorithm uses PID, MPC or LQG control.

9. The solid state closed loop artificial pancreas device of claim 1, wherein said remote electronic display is a smartphone or a wrist watch.

10. The solid state closed loop artificial pancreas device of claim 1, wherein said wireless communications methods include wi-fi, e-mail, texting, cell phone, or Bluetooth.

11. A method of monitoring glucose levels for a patient by using a solid state closed loop artificial pancreas device with LED arrays, comprising:
connecting at least one sensor to measure glucose levels of said patient;
connecting at least one sensor to monitor the physical condition of said artificial pancreas device;
connecting at least one medical device to said patient that affects at least one health parameter of said patient and can be affected by closed-loop feedback;
adding at least one photoswitchable drug formulation whose structure and dosage amounts are controllable by light energy and based on both light wavelength and light intensity to a drug reservoir that receives and holds said photoswitchable drug formulation, can be resupplied through a port, is connected inside the body or outside the body of said patient, and includes at least one set of photoreceivers that detects light from at least one set of LED arrays;
using said sensors and said medical device to generate established values of medically-related conditions and ranges for said patient;
providing said established values of medically-related conditions and ranges for said patient to a wearable computer system that uses closed-loop feedback from said established values and from data provided by said sensors and said medical device to calculate values over a time period and send said values to a physician at regular intervals, to provide hierarchical alerts whenever said sensors or said medical device detects a value outside the established range for said patient or whenever the glucose level of said patient is outside customized values for said patient, and uses a control system algorithm to determine the best and fastest way to bring a value back within range;
connecting at least one set of said LED arrays, controllable by said wearable computer system, to said wearable computer system;
setting the pulse width modulation duty cycle of said LED arrays, or setting the maximum amount of LED activation within a given time period and connected inside the body or outside the body of said patient, so that said LED arrays control the dosage of said photoswitchable drug formulation given to said patient whenever the glucose level of said patient is outside customized values for said patient;
using a set of photoreceivers within said drug reservoir to pass light provided by said LED arrays to said photoswitchable drug formulation so that said photoswitchable drug formulation is dispensed to said patient whenever the glucose level of said patient is outside customized values for said patient;
using said wearable computer program to generate a hierarchy of more than one level of software alerts relating to the health of said patient whenever at least one parameter of said health of said patient is outside established values for said patient;
using said wearable computer program to transmit said software alerts to stakeholders in said health of said patient using wireless communications methods; and,
using a remote electronic display capable of using wireless communications methods to receive said set of software alerts and display said set of software alerts to stakeholders in said health of said patient.

12. The method of claim 11, wherein said photoswitchable drug formulation affects glucose levels of said patient.

13. The method of claim 12, wherein said photoswitchable drug formulation is a diabetes drug formulation, insulin, glucagon, ketones, D10, D50, an anti-inflammatory compound, a chemotherapeutic compound, growth hormones, contraceptives or an analgesic compound.

14. The method of claim 13, wherein said diabetes drug formulation is Metformin, a sulfonylurea compound, a meglitinide, a thiazolidinedione, a DPP-4 inhibitor, a GLP-1 receptor agonist or an SGLT2 inhibitor.

15. The method of claim 14, wherein said sulfonylurea compound is JB253, gliburide, glipizide or glimepiride.

16. The method of claim 11, wherein the LED's within said LED arrays are colored blue or violet.

17. The method of claim 11, wherein said computer system controls said LED arrays and said photoreceivers to operate at different wavelengths of light such that said photoswitchable drug formulations, said LED arrays, said photoreceivers and said wavelengths of light do not interfere with each other.

18. The method of claim 11, wherein said control system algorithm uses PID, MPC or LQG control.

19. The method of claim 11, wherein said remote electronic display is a smartphone or a wrist watch.

20. The method of claim 11, wherein said wireless communications methods include wi-fi, e-mail, texting, cell phone, or Bluetooth.
